(19) United States
(12) Patent Application Publication
(10) Pub. No.: US 2010/0105131 A1
(43) Pub. Date: Apr. 29, 2010

(54) CIRCULATORY PACKED BED REACTOR

(76) Inventor: Qiulan Song, Dongguan (CN)

Correspondence Address: SCIHEAD INTELLECTUAL PROPERTY LAW GROUP, LTD, Huihua Commercial & Trade Building, Suite 1508, 80 Xian Lie Zhong Road, Guangzhou, 51070, CN

(21) Appl. No.: 12/506,271

(22) Filed: Jul. 21, 2009

(30) Foreign Application Priority Data
Oct. 27, 2008 (CN) 200820202419.5

(51) Int. Cl. C12M 1/00

(52) U.S. Cl. 435/289.1

(57) ABSTRACT

An improved circulatory packed bed reactor includes a fermentor, a ventilation system communicating with the fermentor through pipes, and a circulatory system. The fermentor has an upper pipe joint and a lower pipe joint. The circulatory system includes a pipe device and a cell-fixation device. The pipe device includes a circulating pipe outside the fermentor and circulating liquid pumps installed on the circulating pipe. The circulating pipe has one end connecting the upper pipe joint of the fermentor and communicating with the fermentor and another end connecting the lower pipe joint of the fermentor and communicating with the fermentor. The cell-fixation device is located in the fermentor. The cell-fixation device in the fermentor makes the cells in stationary state and submerge in the fermentation to perform deep submerged fermentation, which enhances the density of the fermented cells and cell's tolerance to toxicity, increases the output and the content of cell's secretions, and enables the cells re-utilization.

### BACKGROUND OF THE INVENTION

1. Field of the Invention

The present invention relates to fermentation engineering field and, more particularly, to an improved circulatory packed bed reactor.

2. The Related Art

In microbial engineering, fungal mycelium is usually utilized to be fermented to produce various medicines or health products, such as proteins, polysaccharides, antibiotics, amino acids, etc. Conventional submerged fermentation method is that culture fluid is fermented with free cells. Such method has many disadvantages, for example, the cell density of the liquid fermentation is low, the speed of fermentation reaction is slow, the cell's tolerance to toxicity is poor, and the output and the content of cell production are low. What's more, the cells can not be reused. On the other hand, during liquid fermentation, the fermentation liquid can not contact with fresh air fully and uniformly, so the fermentation liquid is poor at dissolve of oxygen. Moreover, the heat produced by fermentation of cell can not be quickly removed, which will cause cells autolysis. Accordingly, the result of fermentation is undesirable.

### BRIEF SUMMARY OF THE INVENTION

Accordingly, one aspect of the present invention is to provide an improved circulatory packed bed reactor which can make cells fixation in its fermentor, enhance cell density and cell's tolerance to toxicity, increase output and content of cell's secretions, and enable cells re-utilization.

Another aspect of the present invention is to provide an improved circulatory packed bed reactor with inner and outer double circulatory systems which make the fermentation liquid always in circulating state and contact with fresh air fully and uniformly, therefore, the fermentation liquid has a good ability of dissolve of oxygen, and the output and content of cell secretions is increased.

Another aspect of the present invention is to provide an improved circulatory packed bed reactor that can do fermentation repeatedly until the content of the target production in the fermentation liquid meet requirement.

Still another aspect of the present invention is to provide an improved circulatory packed reactor that is advantage in low energy consumption, low cost, and easy operation.

To attain the above objects, the present invention provides an improved circulatory packed bed reactor including a fermentor, a ventilation system communicating with the fermentor through pipes, and a circulatory system. The fermentor has an upper pipe joint and a lower pipe joint. The circulatory system includes a pipe device and a cell-fixation device. The pipe device includes a circulating pipe outside the fermentor and circulating liquid pumps installed on the circulating pipe. The circulating pipe has one end connecting the upper pipe joint of the fermentor and communicating with the fermentor and another end connecting the lower pipe joint of the fermentor and communicating with the fermentor. The cell-fixation device is located in the fermentor.

Preferably, the circulating pipe also includes a liquid-product tube with one end thereof joining and communicating with the circulating pipe and another end thereof serving as a liquid-product exit. A first reversing valve is disposed at the joint of the liquid-product tube and the circulating pipe.

Preferably, the circulating pipe also includes a supplement liquid pipeline with one end there of joining and communicating with the circulating pipe and another end thereof serving as a supplement liquid entrance. A second reversing valve is disposed at the joint of the supplement liquid pipeline and the circulating pipe.

Preferably, the circulatory packed bed reactor further includes a second valve, a cell interceptor and a sampling port mounted on the circulating pipe and located between the lower pipe joint of the fermentor and the circulating liquid pumps in turn.

Preferably, the cell-fixation device includes a centre shaft and at least one layer of spokes mounted on the centre shaft and extanding radially towards outward from the centre shaft. A plurality of cell-fixation carries is mounted on the spokes.

The cell-fixation carries are porous medium.

The fermentor includes a top lid and a side wall. An adjusting speed motor is installed on the outer surface of the top lid. The top end of the centre shaft passes through the top lid of the fermentor and is installed on the adjusting speed motor so that the whole cell-fixation device hangs in the fermentor. At least one runner pipe is installed on the inner surface of the side wall and communicates with the circulating pipe. At least one atomizing spray head is installed on and communicates with the circulating pipe. Liquid in the circulating pipes passes through the runner pipe and the atomizing spray heads and is sprayed to the cell-fixation device by the atomizing spray heads.

Preferably, the circulatory packed bed reactor also includes an autoclave that communicates with the fermentor, the circulatory system and the ventilation system respectively. The autoclave includes an outer wall, an interlayer, an inner wall and an inner chamber. The outer wall and the inner wall are joined together at the top and bottom of the autoclave, respectively. A channel is disposed at the top joint. The autoclave communicates with the upper pipe joint of the fermentor through the channel so as to communicate with the inner chamber of the fermentor. The interlayer is filled with water. The bottom of the autoclave defines an outlet that communicates with the circulating pipe. A venturi tube is installed in the inner chamber of the autoclave. One end of the venture tube opens to the bottom of the autoclave, and the other end of the venture tube passes through the inner wall and the outer wall of the autoclave to communicate with the ventilation system.

Preferably, the channel has a fourth valve mounted thereon whose switch controls the communication and isolation between the fermentor and the autoclave. The channel defines a channel pipe joint between the lower pipe joint of the fermentor and the fourth valve. The channel pipe joint joins and communicates with the circulating pipes, and a third reversing valve is disposed at the joint of the channel pipe joint and the circulating pipe.

Preferably, the circulatory packed bed reactor further includes a ventilation pipe connecting the fermentor and the inner chamber of the autoclave. A sixth valve and a seventh valve are mounted on the ventilation pipe. The circulatory packed bed reactor also includes a pressure detecting device communicating with the interlayer of the autoclave and the ventilation pipe at a point between the sixth valve and the seventh valve, respectively.

The advantages of the present invention are as follows: (1) the cell-fixation device in the fermentor makes the cells in stationary state and submerge in the fermentation to perform deep submerged fermentation, which enhances the density of the fermented cells and cell's tolerance to toxicity and increases the output and the content of cell's secretions, what's more, the fixed cells can be reused. (2) The double circulatory systems make the fermented liquid circulation in the fermentor, that's to say, one part of the fermented liquid is used to yield new production constantly, another part of the fermented liquid is mixed with fresh culture fluid, then the mixture is fed in the reactor at a constant speed. Thus the reatctor can not only continue producing but also improve the content of production in the fermented liquid. Furthermore, the culture being fed in the reactor makes the fixed cells grow under flow condition, which improves the transfer of heat and the content of dissolved oxygen in the reactor which in turn, benefits to cell growth and propagation and biosynthesis of production.

### BRIEF DESCRIPTION OF THE DRAWINGS

The present invention will be apparent to those skilled in the art by reading the following description of preferred embodiments thereof, with reference to the attached drawings, in which:

FIG. 1 is a schematic diagram of an improved circulatory packed bed reactor of a first embodiment in accordance with the present invention;

FIG. 2 is a top view of a cell-fixation device of the improved circulatory packed bed reactor with a lid thereof removed;

FIG. 3 is a schematic diagram of the improved circulatory packed bed reactor of a second embodiment in accordance with the present invention;

FIG. 4 illustrates the circular flow direction of the cell stock solution in the improved circulatory packed bed reactor of the first embodiment shown in FIG. 1;

FIG. 5 illustrates the circular flow direction of the culture fluid or the mixture of the culture fluid and the fermentation liquid in the improved circulatory packed bed reactor of the first embodiment shown in FIG. 1;

FIG. 6 illustrates the flow direction of the finished product in the improved circulatory packed bed reactor of the first embodiment shown in FIG. 1;

FIG. 7 illustrates the flow direction of the culture fluid in the improved circulatory packed bed reactor of the second embodiment shown in FIG. 3;

FIG. 8 illustrates the circular flow direction of the cell stock solution in the circulatory improved packed bed reactor of the second embodiment shown in FIG. 3;

FIG. 9 illustrates the circular flow direction of the culture fluid or the mixture of the culture fluid and the fermentation liquid in the improved circulatory packed bed reactor of the second embodiment shown in FIG. 3; and

FIG. 10 illustrates the flow direction of the finished product in the improved circulatory packed bed reactor of the second embodiment shown in FIG. 3.

### DETAILED DESCRIPTION OF THE PREFERRED EMBODIMENTS

For facilitating understanding, like components are designated by like reference numerals throughout the various embodiments of the invention as shown in the attached drawings.

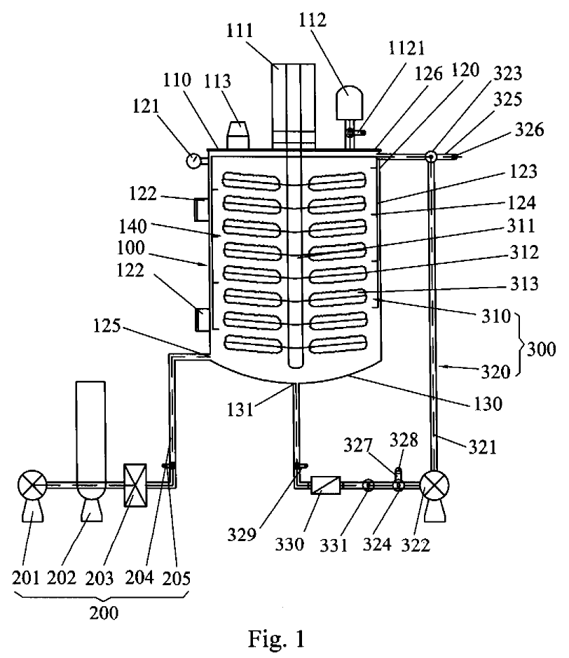

FIG. 1 illustrates an improved circulatory packed bed reactor of a first embodiment in accordance with the present invention, including a fermentor 100, a ventilation system 200, and a circulatory system 300.

The fermentor 100 includes a top lid 110, a side wall 120, a bottom 130 and a inner chamber 140. An adjustable speed motor 111, a breather valve 112 and a sight glass light 113 are installed on the outer surface of the top lid 110. A first valve 1121 is disposed on the breather valve 112 and controls the open and close of the breather valve 112. A pressure detecting device 121 and two sight glasses 122 are installed on the outer surface of the side wall 120. One or several runner pipes 123 are installed on the inner surface of the side wall 120 and communicate with the circulatory system 300. One or several anodize spay heads 124 are installed on the runner pipes 123 and communicates with the circulatory system 300. A ventilation outlet 125 is installed on the sidewall 120 and communicates with the ventilation system 200. A pipe outlet 126 is installed on the upper of the fermentor and communicates with the circulatory system 300. A lower pipe joint 131 is installed on the bottom of fermentor 130 and also communicates with the circulatory system 300.

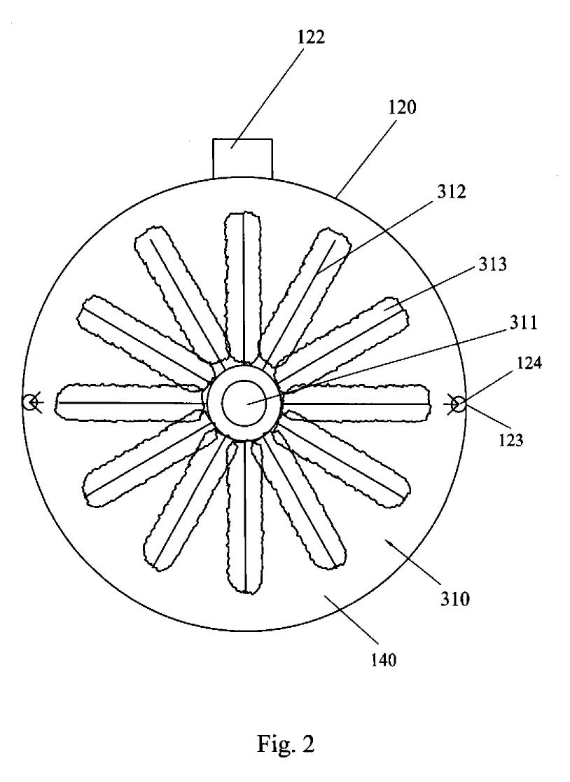

The circulatory system 300 includes a cell-fixation device 310 and a pipe device 320. Referring to FIG. 2 in conjunction with FIG. 1, the cell-fixation device 310 includes a centre shaft 311 and a plurality of spokes 312 mounted on the centre shaft 311 and extending radially towards outward from the central of the centre shaft 311. A plurality of cell-fixation carries 313, which are porous medium, are mounted on the spokes 312. The centre shaft 311 passes through the top lid 110 of the fermentor and is installed on the adjustable speed motor 111, thus the whole cell-fixation device 310 hangs in the fermentor The adjustable speed motor 111 controls the turning speed of the cell-fixation device 310. The anodize spay heads 124 spay culture fluid or fermented liquid from the circulatory system 300 to the cell-fixation device 310.

The pipe device 320 includes a circulating pipe 321 outside the fermentor and circulating liquid pumps 322 installed on the circulating pipe 321. The circulating pipe 321 has one end connecting the upper pipe joint 126 of the fermentor 100 and communicating with the fermentor 100, and another end connecting the lower pipe joint 131 of the fermentor 100 and communicating with the fermentor 100.

The circulating pipe 321 also includes a liquid-product pipeline 325 with one end thereof joining and communicating with the circulating pipe 321 and another end thereof serving as a liquid-product exit 326. A first reversing valve 323 is disposed at the joint of the liquid-product exit 326 and the circulating pipe 321.

The circulating pipe 321 also includes a supplement liquid pipeline 327 with one end thereof joining and communicating with the circulating pipe 321 and another end thereof serving as a supplement liquid entrance 328. A second reversing valve 324 is disposed at the joint of the supplement liquid entrance 328 and circulating pipe 321.

It is appreciated that, alternately, the supplement liquid entrance 328 can also be disposed directly on the upper part of the fermentor 100, the liquid-product exit 326 is disposed directly on the lower part of the fermentor 100, or the supplement liquid entrance 328 is disposed directly on the upper part of the fermentor 100, the liquid-product exit 326 communicates with the circulating pipe 321 through the liquid-product pipeline 325 and a valve 323 is disposed at the joint, or the liquid-product exit 326 is disposed at the lower part of the fermentor 100, the supplement liquid entrance 328 communicates with the circulating pipe 321 through the supplement liquid pipeline 327, and a second reversing valve 324 is disposed at the joint.

A second valve 329, a cell interceptor 330, and a sampling port 331 are mounted on the circulating pipe 321. The second valve 329, the cell interceptor 330, and the sampling port 331 are located between the lower pipe joint 131 under the fermentor 100 and the second reversing valve 324 in turn.

The ventilation system 200 includes a roots blower 201, an air filtrating system 202, an air cool system 203, a ventilation pipe 204, and a third valve 205 on the ventilation pipe 204. The roots blower 201, the air filtrating system 202, and the air cool system 203 communicate with each other through pipe in turn. The ventilation pipe 204 communicates with the ventilation outlet 125 on the side wall of the fermentor 100, and air is transferred to the fermentor 100 through the ventilation pipe 204.

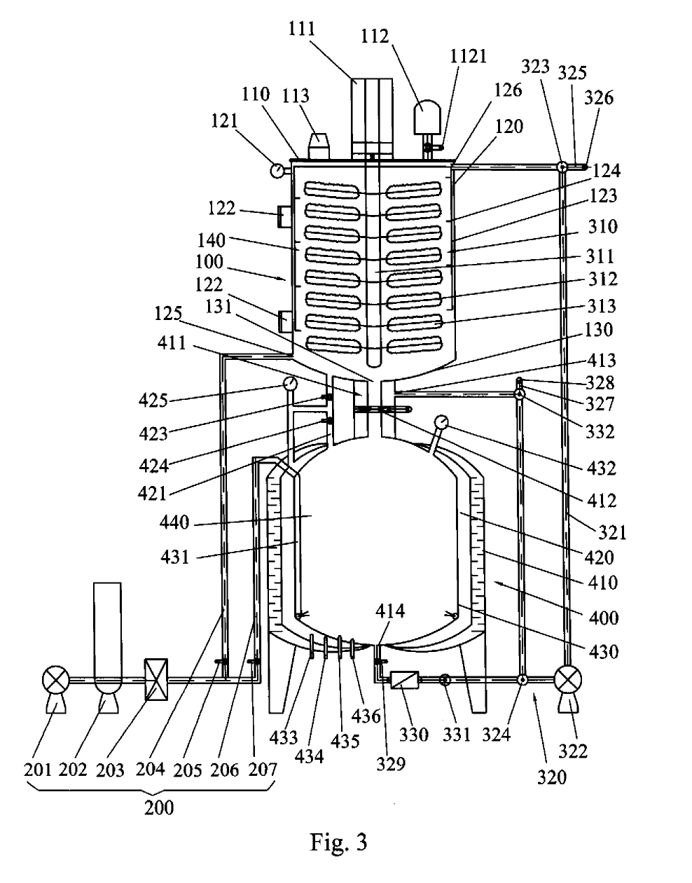

FIG. 3 illustrates an improved circulatory packed bed reactor of a second embodiment in accordance with the present invention. The reactor includes an autoclave 400 communicating with the fermentor 100, the ventilation system 200, and the circulating pipe 300, respectively. The autoclave 400 includes an outer wall 410, an interlay 420, an inner wall 430, and an inner chamber 440. The outer wall 410 and the inner wall 430 are joined together at the top and the bottom of the autoclave 400, respectively. A channel 411 is disposed between the top joint and the lower pipe joint 131 at the bottom 130 of the fermentor 100 so that the inner chamber 440 of the autoclave 400 communicates with the inner chamber 140 of the fermentor 100 through the channel 411. A fourth valve 412 is disposed on the channel 411. The opening and closing of the fourth valve 412 control the communication and isolation of the inner chamber 140 of the fermentor 100 and the inner chamber 440 of the autoclave 400. A channel pipeline joint 413 is disposed on the channel 411 and is located between the down pipe joint 131 of the fermentor 100 and the fourth valve 412. The channel pipeline joint 413 communicates with the supplement liquid pipeline 327 through circulating pipe 321 and passes through the second valve 332. At the bottom of the autoclave 400 has an outlet 414. The outlet 414 communicates with the bottom of the circulating pipe 312. A venturi tube 431 is installed in the inner chamber 440 of the autoclave 400. One end of the venturi tube 431 opens to the bottom of the autoclave 400, the other end of venturi tube 431 passes through the inner wall and the outer wall of the autoclave 100 to communicate with the ventilation system 200. A pressure detecting device 432 is installed on the inner wall 430 of the autoclave 400 to detect the pressure of the inner chamber 440.

On the ventilation pipeline 206 has a fifth valve 207 which controls whether transfer air to the inner chamber 440 of the autoclave 400 or not. The ventilation pipeline 206 communicates with the ventilation pipeline 204 between the air cool system 203 and the third valve 205.

The interlay 420 communicates with the inner chamber 140 of the fermentor 100 through a ventilation pipeline 421. A sixth valve 423 and a seventh valve 424 are disposed on the ventilation pipeline 421. A pressure detecting device 425 communicates with the interlay 420 and the ventilation pipeline 421 between the sixth valve 423 and the seventh valve 424, respectively, and detects the pressure in the interlay 420 of the autoclave 400. The autoclave 400 also has an electric device 433. The interlay 420 is filled with water. The electric device 433 heats the water in the interlay 420 and sterilizes the inner chamber 440 of the autoclave 400. When the sixth valve 423 and the seventh valve 424 on the ventilation pipeline 421 are opening, the inner chamber 140 of the fermentor 100 is also sterilized. When the sixth valve 423 is closed while the seventh valve 424 is opening, only the inner chamber 440 is sterilized. The autoclave 400 also has a pressure detecting device 432, a temperature detecting device 434, a pH detecting device 435 and a dissolved oxygen detecting device 436, all of those serve to detect the parameters of the fermented liquid in the inner chamber 440.

The working process of the present circulatory packed bed reactor will be described in great detail hereinafter:

The working process of the first embodiment of the circulatory packed reactor:

1. Sterilization:

Open the top lid 110 of the fermentor 100; install the cell-fixation carries 313 on the spokes 312 of the cell-fixation device 310; close the top lid 110; and sterilize the reactor.

2. Inoculation:

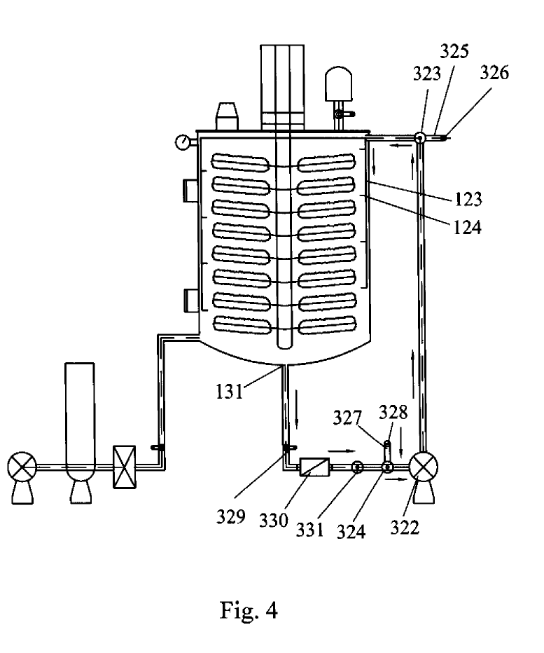

Referring to FIG. 4 in which the arrows show the flow direction of cell stock solution.

When the reactor is cooled at room temperature, input the prepared cell stock solution into the supplement liquid pipeline 328 through the supplement liquid entrance 327; rotate the second reversing valve 324; cause the cell stock solution to flow towards the cycle liquid bump 322 by the second valve 324; with the power provided by the cycle liquid bump 322, the cell stock solution flows toward the first reversing valve 323 through circulating pipe; rotate the first reversing valve 323; cause the cell stock solution to flow towards the runner pipeline 123 in the fermentor 100 through pipeline and through the runner pipeline 123 to the anodize spay heads 124; the anodize spay heads 124 spay the cell stock solution to the cell-fixation carries 313 on which the cell is fixed. The cell stock solution gathers at the bottom 130 of the fermentor, passes through the lower pipe joint 131 in the bottom 130 of the fermentor into the circulating pipe 321 again, then passes through the second valve 329, the cell interceptor 330, the sampling port 331 and the second reversing valve 324 in turn, and finally enters another circulation. The cell stock solution flows circularly and causes an amount of cells fixation on the cell-fixation carries 313. When the amount of fixed cells achieves the predetermined value, stop the incubation.

In the process, the flow direction of the cell stock solution is as follow: the supplement liquid entrance 328—the supplement liquid pipeline 327—the second reversing valve 324—the cycle liquid bump 322—the first reversing valve 323—the runner pipeline 123—the anodize spay heads 124—the cell-fixation carries 313—the lower pipe joint 131 in the bottom 130 of the fermentor—the second valve 329—the cell interceptor 330—the sampling port 331—the second reversing valve 324—the cycle liquid bump 322

3. The First Fermentation Incubation:

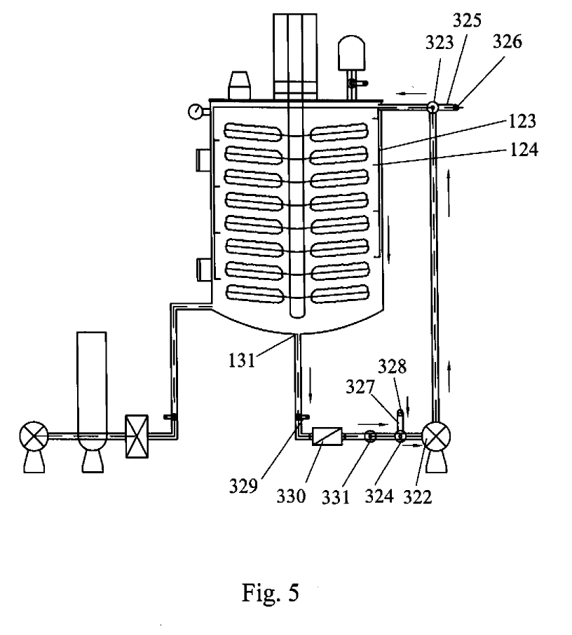

Referring to FIG. 5 in which the arrows show the flow direction of the culture fluid.

After the culture fluid is sterilized, input the culture fluid into the supplement liquid pipeline 327 through the supplement liquid entrance 328; rotate the second reversing valve 324; cause the culture fluid to flow towards the cycle liquid bump 322 through the second reversing valve 324; with the power provided by the cycle liquid bump 322, the culture fluid flows towards the first reversing valve 323 through circulating pipe; rotate the first reversing valve 323; cause the cell stock solution to flow towards the runner pipeline 123 in the fermentor 100 through pipeline and through the runner pipeline 123 to the anodize spay heads 124; the anodize spay heads 124 spay the cell stock solution to the cell-fixation carries 313 on which the cell is fixed. The culture fluid gathers at the bottom 130 of the fermentor, passes through the lower pipe joint 131 in the bottom 130 of the fermentor into circulating pipe 321 again, then passes through the second valve 329, the cell interceptor 330, the sampling port 331 and the second reversing valve 324 in turn, and finally enters another circulation.

The flow direction of the culture fluid is as follow: the supplement liquid entrance 328—the supplement liquid pipeline 327—the second reversing valve 324—the cycle liquid bump 322—the first reversing valve 323—the runner pipeline 123—the anodize spay heads 124—the cell-fixation carries 313—the lower pipe joint 131 in the bottom 130 of the fermentor—the second valve 329—the cell interceptor 330—the sampling port 331—the second reversing valve 324

4. Circulatory Fermentation and Incubation and Collection Part of Production at the Same Time:

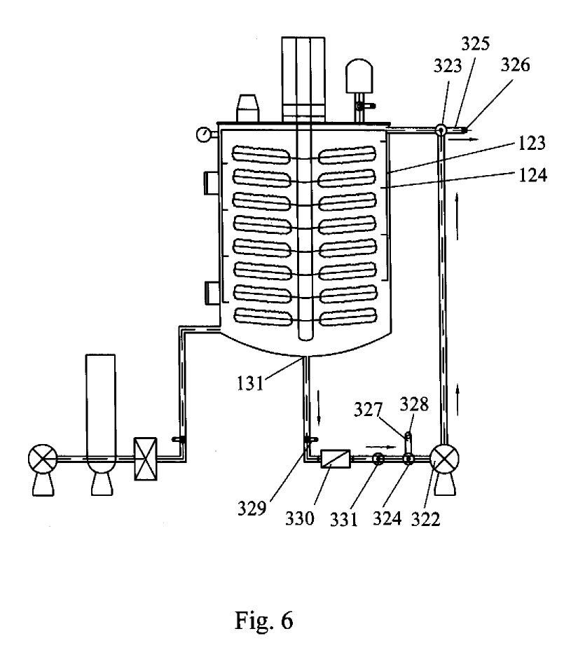

Referring to FIG. 6 in which the arrows show the flow direction of the production.

Collect production: when the cells are fermented for some time, take up a little sample from the sampling port 331; detect the content in the fermented liquid. If the content attains the target value, stop the first circulatory fermentation. Then rotate the first reversing valve 323 to lead the fermented liquid to flow towards the liquid-product entrance 326, and collect the production.

The flow direction of the liquid-product is as follow: the lower pipe joint 131—the second valve 329—the cell interceptor 330—the sampling port 331—the second reversing valve 324—the cycle liquid bump 322—the first reversing valve 323—the liquid-product pipeline 325—the liquid-product entrance 326.

Circulatory fermentation: Please refer back to FIG. 5 in which the arrows show the flow direction of the mixture liquid. While collecting the production, retain parts of the fermented liquid in the fermentor 100; supplement fresh culture into the fermentor; after the fresh culture is sterilized, input the fresh culture into the supplement liquid pipeline 327 through the supplement liquid entrance 328; rotate the second reversing valve 324; lead the culture to flow towards the cycle liquid bump 322 through the second reversing valve 324; and with the power provided by the cycle liquid bump 322, the culture flow towards the first reversing valve 323 through circulating pipe; rotate the first reversing valve 323; lead the culture to flow towards the runner pipeline 123 in the fermentor 100 through pipeline and through the runner pipeline 123 to the anodize spay heads 124; the anodize spay heads 124 spay the culture to the cell-fixation carries 313. The culture fluid gathers at the bottom 130 of the fermentor and mixes with the retain fermenting liquid. The mixture passes through the lower pipe joint 131 in the bottom 130 of the fermentor into circulating pipe 321 again, then passes through the second valve 329, the cell interceptor 330, the sampling port 331 and the second reversing valve 324 in turn, and finally enters another circulation.

The flow direction of the mixture is as follow: the culture fluid—the supplement liquid entrance 328—the supplement liquid pipeline 327—the second reversing valve 324—the cycle liquid bump 322—the first reversing valve 323—the runner pipeline 123—the anodize spay heads 124—the cell-fixation carries 313—the lower pipe joint 131 in the bottom 130 of the fermentor—the second valve 329—the cell interceptor 330—the sampling port 331—the second reversing valve 324.

Repeat the steps 3 and 4, until the content in the fermented liquid attains target value.

5. Collect the Production: Collect the Production and Stop Fermentation.

It is noted that in the whole process of the cell's growth and fermentation, the ventilation system feeds air through the pipeline into the fermentor 100 for the cell's growth and discharges the exhaust gas through the breather valve all the time.

The working process of the second embodiment of the circulatory packed bed reactor:

1. Sterilization:

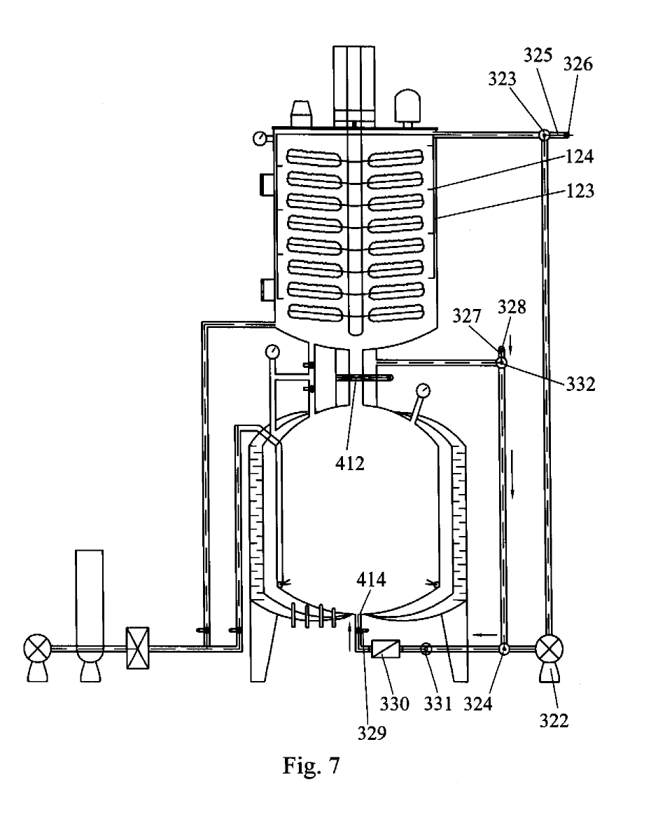

Referring to FIG. 7 in which the arrows show the flow direction of the culture.

Open the top lid 110 of fermentor 100; install cell-fixation carries 313 on the radius 312 of cell-fixation device 310; close the top lid 110; open the valve 423 and the valve 424 on the ventilation pipeline 421; make the interlay 420 of the autoclave communicate with the inner chamber 140 of the fermentor; input the prepared culture into the supplement liquid pipeline 327 through the supplement liquid entrance 328; rotate the second reversing valve 324 and the third reversing valve 332; open the second valve 329; lead the culture to flow into the circulating pipe through the third reversing valve and flow towards the sampling port 331 through the second reversing valve 324. The culture passes through the sampling port 331 and the cell interceptor 330, reaches the outlet 414 at the bottom of the fermentor, and enters the inner chamber 440 of the autoclave 400. Then turn on the electric device 450 to sterilize the inner chamber 140 of the fermentor 100, the inner chamber 440 of the autoclave 400, the cell-fixation carries 313 in the inner chamber 140, and the culture.

The flow direction of the culture fluid: the supplement liquid entrance 328—the supplement liquid pipeline 327—the third reversing valve 332—the second reversing valve 324—the sampling port 331—the cell interceptor 330—the second valve 329—the outlet 414 at the bottom of the fermentor 100—the inner chamber 440 of the autoclave 400.

2. Inoculation:

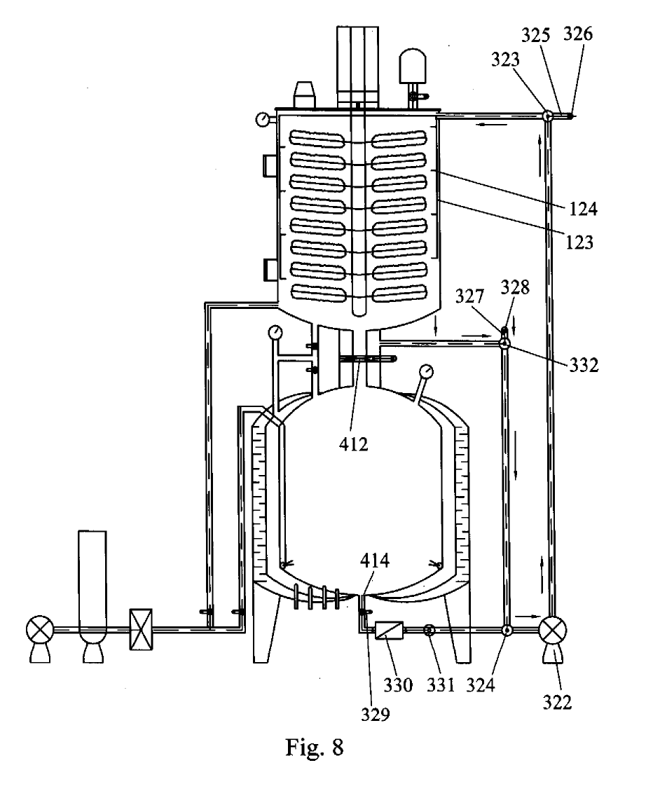

Referring to FIG. 8 in which the arrows show the flow direction of cell stock solution.

When the reactor is cooled at room temperature, close the valve 423 and the valve 424 on the ventilation pipeline 421; make the inner chamber 140 of the fermentor 100 isolate with the interlay 430 and the inner chamber 440 of the autoclave 100; input the prepared cell stock solution into the supplement liquid pipeline 327 through the supplement liquid entrance 328; rotate the third reversing valve 332 and the second reversing valve 324; lead the cell stock solution to flow towards the cycle liquid bump 322 through the third valve 332 and the second valve 324; with the power provided by the cycle liquid bump 322, the cell stock solution flows toward the first reversing valve 323 through circulating pipe; rotate the first reversing valve 323; lead the cell stock solution to flow towards the runner pipeline 123 in the fermentor 100 through pipeline and through the runner pipeline 123 to the anodize spay heads 124; the anodize spay heads 124 spay the cell stock solution to the cell-fixation carries 313 on which the cell is fixed. The cell stock solution gathers at the bottom 130 of the fermentor 100, passes through the channel 411 to flow towards the third reversing valve 332, and finally enters another circulation. The cell stock solution flows circularly to cause an amount of cells fixation on the cell-fixation carries 313. When the amount of cell inoculation attains the predetermined value, stop the circulatory.

The flow direction of the cell stock solution is as follow: the supplement liquid entrance 328—the supplement liquid pipeline 327—the third reversing valve 332—the second reversing valve 324—the cycle liquid bump 322—the first reversing valve 323—the circulating pipeline 123—the anodize spay heads 124—the cell-fixation carries 313—the bottom 130 of the fermentor 100—the channel 411—the third reversing valve 332.

3. The First Time Fermentation and Incubation:

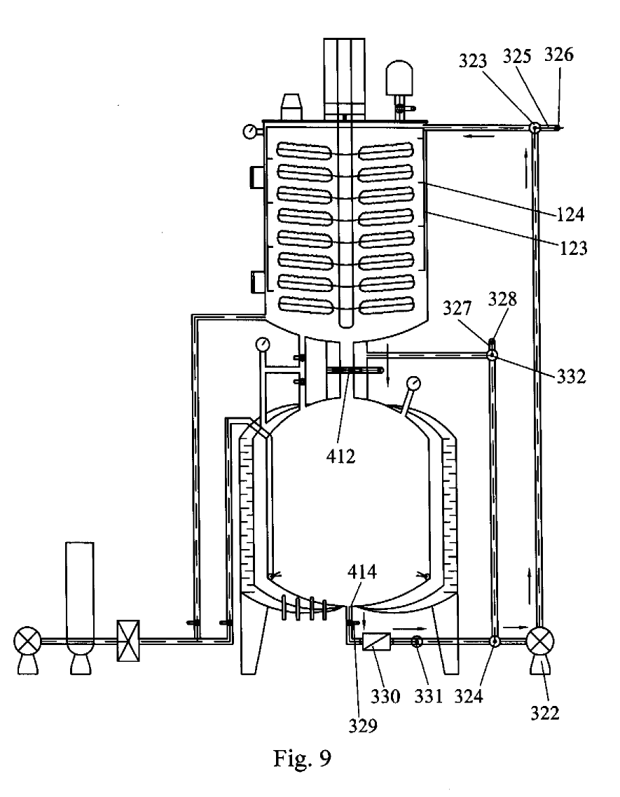

Referring to FIG. 9 in which the arrows show the flow direction of the culture fluid.

When the culture is cooled at room temperature, prepare for fermentation. Open the second valve 329 and the valve 412 on the channel 411; the culture flows out from the outlet 414 at the bottom 414 of the autoclave, passes through the second valve 329, the cell interceptor 330 and the sampling port 331 in turn, and attains the second reversing valve 324; rotate the second reversing valve 324; cause the culture fluid to flow towards the cycle liquid bump 322 through the pipelines; with the power provided by the cycle liquid bump 322, the culture fluid flow towards the first reversing valve 323 through circulating pipe; rotate the first reversing valve 323; lead the culture fluid to flow towards the circulating pipeline 123 in the fermentor 100 through pipeline and through the runner pipeline 123 to the anodize spay heads 124; the anodize spay heads 124 spay the culture fluid to the cell-fixation carries 313. The culture fluid gathers at the bottom 130 of the fermentor, and passes through the channel 411 into the inner chamber 140 of the autoclave 400, then passes through the outlet 414 at the bottom of the autoclave 400, and finally enters another circulation. For prevent the cell from choking the outlet 414 in the circulatory, open the valve 207 and transfer air to the venturi tube 332 through the ventilation pipeline 206, which causes the liquid in the inner chamber 430 to roll, thus avoid the outlet 414 to be choked.

The flow direction of the fermented liquid: the culture fluid—the outlet 414—the second valve 329—the cell interceptor 330—the sampling port 331—the second reversing valve 324—the cycle liquid bump 322—the first reversing valve 323—the runner pipeline 123—the anodize spay heads 124—the cell-fixation carries 313—the bottom 130 of the fermentor 100—the channel 411—the inner chamber 440 of the autoclave 400—the outlet 414.

4. The Second Time Circulatory Fermentation and Incubation:

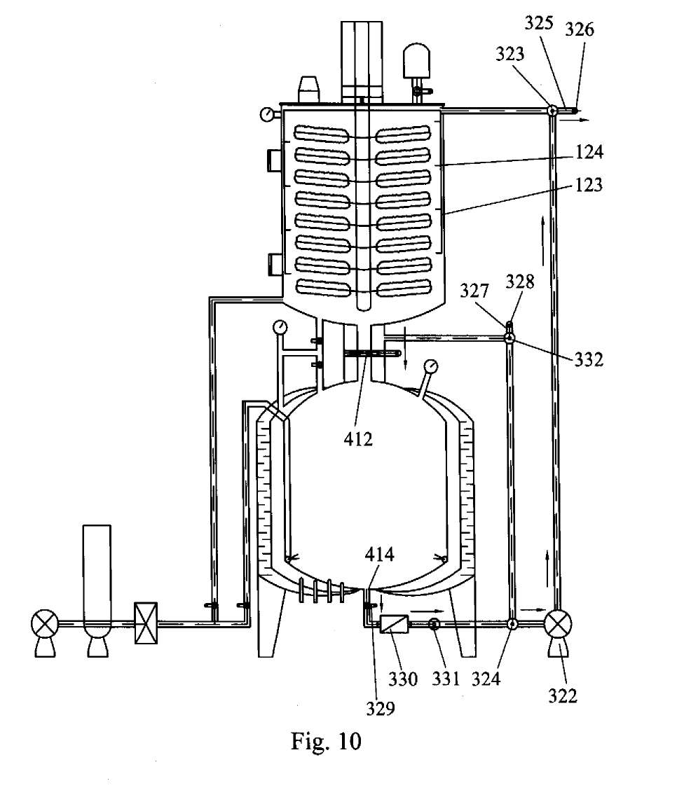

Referring to FIG. 10 in which the arrows show the flow direction of the production.

Collect the production: when the cells are fermented for some time, take up a litter sample from the sampling port 331 and detect the content in the fermented liquid. If the content attains the target value, stop the first circulatory fermentation. Then close the valve 412 on the channel 411; isolate the inner chamber 140 of the fermentor 100 and the inner chamber 440 of the autoclave 400; rotate the first reversing valve 323; lead the fermented liquid to flow towards the production outlet 326; and collect the production.

The flow direction of the production: the production—the outlet 414—the second valve 329—the cell interceptor 330—the sampling port 331—the second reversing valve 324—the cycle liquid bump 322—the first reversing valve 323—the production pipelines 325—the production outlet 326.

circulatory fermentation: please also refer to FIG. 9 in which the arrows show the flow direction of the mixture. Retain part of the fermented liquid; rotate the first reversing valve 323; lead the fermented liquid to flow towards the fermentor 100 and store at the fermentor 100. When the fermented liquid in the inner chamber 440 of the autoclave 440 flows out completely, rotate the third reversing valve 332 and the second reversing valve 324; input the prepared culture fluid into the supplement outlet 328; lead the culture fluid to flow towards the cell interceptor 330 through the third reversing valve 332 and the second reversing valve 324 and into the inner chamber 440 of the autoclave 400 and sterilize the culture fluid; cool the culture fluid to room temperature; open the valve 412 on the channel 411; the retained fermented liquid is mixed with the culture fluid; rotate the second valve 324 and the first valve 323; the mixture passes through the outlet 414 of the autoclave, the cell interceptor 330, the sampling port 331, the second reversing valve 324, the cycle liquid bump 322, and the first reversing valve 323, flows into the runner pipeline 123 in the fermentor 100 through pipeline and through the runner pipeline 123, and reaches to the anodize spay heads 124; the anodize spay heads 124 spay the culture on the cell-fixation carries; and finally enters another circulation.

The flow direction of the mixture is as follow: in the autoclave, the retained fermented liquid in the fermentor is mixed with the culture fluid in the autoclave—the outlet 414—the second valve 329—the cell interceptor 330—the sampling port 331—the second reversing valve 324—the cycle liquid bump 322—the first reversing valve 323—the runner pipeline 123—the anodize spay heads 124—the cell—fixation carries 313—the channel 441—the inner chamber 140 of autoclave 400—the outlet 414.

Repeat the steps 3 and 4, until the content in the fermented liquid attains target value.

5. Collect the Production: Collect the Production and Stop Fermentation.

It is noted that in the whole process of the cell's growth and fermentation, the ventilation system feeds air through the pipeline into the fermentor 100 for the cell's growth and discharges the exhaust gas through the breather valve all the time.

The foregoing description of the present invention has been presented for purposes of illustration and description. It is not intended to be exhaustive or limit the invention to the precise form disclosed, and obviously many modifications and variations are possible in light of the above teaching. Such modifications and variations that may be apparent to those skilled in the art are intended to be included within the scope of this invention as defined by the accompanying claims.

What is claimed is:

1. A circulatory packed bed reactor including a fermentor and a ventilation system communicating with said fermentor through pipes, said fermentor having an upper pipe joint and a lower pipe joint, the circulatory packed bed reactor characterized in that: the circulatory packed bed reactor further includes a circulatory system; said circulatory system includes a pipe device and a cell-fixation device, said pipe device includes a circulating pipe outside the fermentor and circulating liquid pumps installed on said circulating pipe; said circulating pipe has one end connecting the upper pipe joint of the fermentor and communicating with the fermentor and another end connecting the lower pipe joint of the fermentor and communicating with the fermentor; and said cell-fixation device is located in said fermentor.

2. The circulatory packed bed reactor of claim 1, further including a liquid-product pipeline with one end thereof joining and communicating with said circulating pipe and another end thereof serving as a liquid-product exit, and a first reversing valve is disposed at the joint of the liquid-product pipeline and the circulating pipe.

3. The circulatory packed bed reactor of claim 1, further including a supplement liquid pipeline with one end thereof joining and communicating with said circulating pipe and another end thereof serving as a supplement liquid entrance, and a second reversing valve is disposed at the joint of said supplement liquid pipeline and said circulating pipe.

4. The circulatory packed bed reactor of claim 1, further including a second valve, a cell interceptor and a sampling port mounted on said circulating pipe, and said second valve, said cell interceptor and said sampling port being located between said lower pipe joint of the fermentor and said circulating liquid pumps in turn.

5. The circulatory packed bed reactor of claim 1, wherein said cell-fixation device includes a centre shaft and at least one layer of spokes mounted on the center shaft and extending radially towards outward from the centre shaft, and a plurality of cell-fixation carries is mounted on the spokes.

6. The circulatory packed bed reactor of claim 5, wherein said cell-fixation carries are porous medium.

7. The circulatory packed bed reactor of claim 5, wherein said fermentor includes a top lid and a side wall; an adjustable speed motor is installed on the outer surface of said top lid, a top end of said centre shaft passes through the top lid of the fermentor and is installed on said adjustable speed motor so that the whole cell-fixation device hangs in the fermentor; at least one runner pipe is installed on the inner surface of the side wall and communicates with the circulating pipe, at least one atomizing spray head is installed on and communicates with said circulating pipe, liquid in said circulating pipe passes through said runner pipe and said atomizing spray head and is sprayed to said cell-fixation device by said atomizing spray head.

8. The circulatory packed bed reactor of claim 1, further including a autoclave that communicates with said fermentor, said circulatory system and said ventilation system, respectively; wherein said autoclave includes an outer wall, an interlayer, an inner wall and an inner chamber; said outer wall and said inner wall are joined together at the top and the bottom of said autoclave, respectively; a channel is disposed at the top joint, said autoclave communicates with said upper pipe joint of said fermentor through said channel so as to communicate with the inner chamber of said fermentor; said interlayer is filled with water; the bottom of said autoclave defines an outlet, said outlet communicates with said circulating pipe; a venturi tube is installed in the inner chamber of said autoclave, one end of said venturi tube opens to the bottom of said autoclave, and the other end of said venturi tube passes through the inner wall and the outer wall of said autoclave to communicate with the ventilation system.

9. The circulatory packed bed reactor of claim 8, wherein said channel has a fourth valve mounted thereon whose switch controls the connectivity and isolation between said fermentor and said autoclave, said channel defines a channel pipe joint between the lower pipe joint of said fermentor and said fourth valve, said channel pipe joint joins and communicates with said circulating pipe, and a third reversing valve is disposed at the joint of said channel pipe joint and said circulating pipe.

10. The circulatory packed bed reactor of claim 8, further including a ventilation pipe connecting said fermentor and said inner chamber of said autoclave, said ventilation pipe having a sixth valve and a seventh valve mounted thereon, and further including a pressure detecting device communicating with said interlayer of said autoclave and said ventilation pipe at a point between said sixth valve and said seventh valve, respectively.
